(19) United States
(12) Patent Application Publication
(10) Pub. No.: US 2003/0186315 A1
(43) Pub. Date: Oct. 2, 2003

(54) ADMINISTRATION OF AN AGENT WHICH DECREASES PROCESSING OF AMYLOID PRECURSOR PROTEIN

(76) Inventors: VIRGINIA M.-Y. LEE, PHILADELPHIA, PA (US); ROBERT W. DOMS, BERWYN, PA (US)

Correspondence Address: JANE MASSEY LICATA, LAW OFFICES OF JANE MASSEY LICATA, 66 E MAIN STREET, MARLTON, NJ, 08053

(21) Appl. No.: 09/297,877

(22) PCT Filed: Nov. 13, 1997
(86) PCT No.: PCT/US97/20212

(51) Int. Cl. G01N 33/53

(52) U.S. Cl. 435/7.1

(57) ABSTRACT

β secretase pathways which process amyloid precursor protein into the amyloid peptides of neuritic plaques and vascular deposits that accumulate in the brains of patients with Alzheimer's disease have been identified in the endoplasmic reticulum of neuronal cells. Methods of identifying compounds which increase or decrease processing of amyloid precursor protein via these pathways which may be useful in the treatment of Alzheimer's disease and in identifying causative agents of this disease are provided.

### INTRODUCTION

This invention was made in the course of research sponsored by the National Institutes of Health. The U.S. Government may have certain rights in this invention.

### FIELD OF THE INVENTION

Patients suffering from Alzheimer's disease are afflicted with neuritic plaques and vascular deposits consisting of amyloid fibrils made up of amyloid A peptides. Novel β-secretase enzymatic pathways which process amyloid β precursor peptide to amyloid β peptides have now been identified and are located in the endoplasmic reticulum. The present invention relates to methods of identifying agents which increase or decrease processing of amyloid precursor protein into amyloid β peptides by contacting NT2N cells with the agent and measuring levels of amyloid β peptides formed in the endoplasmic reticulum of the cells. Agents identified to increase processing of amyloid precursor protein into amyloid β peptides can be used in the early diagnosis of Alzheimer's disease while agents identified to decrease this processing are expected to be useful in the treatment of Alzhiemer's disease.

### BACKGROUND OF THE INVENTION

Amyloid β (Aβ) peptides are the building blocks of the amyloid fibrils found in neuritic plaques and vascular deposits that accumulate in the brains of patients with Alzheimer's disease (AD; Selkoe, D. J. 1994 Ann. Rev. Cell. Biol. 10:373-403). Aβ is derived from proteolytic processing of one or more isoforms of the amyloid precursor protein (APP; Kang et al. 1987 Nature 325:733-736). APP isoforms are alternatively spliced type I transmembrane glycoproteins that are encoded by a single gene on human chromosome 21 (Kang et al. 1987 Nature 325:733-736; St. George-Hyslop et al. 1987 Science 235:885-890). The 39-43 amino acid long Aβ sequence begins in the ectodomain of APP and extends into the transmembrane region. Of the 3 major Aβ-containing isoforms encoded by the APP gene (i.e., APP695, APP751, and APP770; Kang et al. 1987 Nature 325:733-736; Ponte et al. 1988 Nature 331:525-527; Tanzi et al. 1988 Nature 331:528-530; Kitaguchi et al. 1988 Nature 331:530-532), APP695 is expressed almost exclusively by neurons of the central and peripheral nervous systems (Golde et al. 1990 Neuron 4:253-267; Kang, J. and Müller-Hill, B. 1990 Biochem. Biophys. Res. Commun. 166:1192-1200; Arai et al. 1991 Ann. Neurol. 30:686-693).

Newly synthesized APP matures in the endoplasmic reticulum and the Golgi apparatus acquiring N- and O-linked carbohydrates, tyrosine sulfates (Weidemann et al. 1997 Nature Med. 3:328-332; Oltersdorf et al. 1990 J. Biol. Chem. 265:4492-4497) and phosphates (Oltersdorf et al. 1990 J. Biol. Chem. 265:4492-4497; Suzuki et al. 1992 Neurosci. 48:755-761; and Knops et al. 1993 Biochem Biophy. Res. Comm. 197:380-385). Several pathways of APP metabolism have been described in cultured cells, and evidence suggests that the relative importance of each pathway depends on the cell type. For example, non-neuronal cells preferentially process APP by the α-secretase pathway which cleaves APP within the Aβ sequence, thereby precluding the formation of Aβ (Esch et al. 1990 Science 248:1122-1124; Sisodia et al. 1990 Science 248:492- 495). The putative α-secretase enzyme(s) is active at or near the cell surface, causing the N-terminal fragment (APPα) to be quickly secreted. In contrast, neuronal cells process a much larger portion of APP by the β-secretase pathway(s), which generates intact Aβ by the combined activity of two enzyme classes. The β-secretase(s) cleaves APP at the amino terminus of the Aβ domain releasing a distinct N-terminal fragment (APPβ). In addition, the T-secretase(s) cleaves APP at alternative sites of the carboxy terminus generating species of Aβ that are either 40 (Aβ₄₀) or 42 amino acids long (Aβ₄₂; Seubert et al. 1993 Nature 361:260-263; Suzuki et al. 1994 Science 264:1336-1340; Turner et al. 1996 J. Biol. Chem. 271:8966-8970).

Aβ₄₂ is believed to have a significant role in AD pathology. Studies have shown that Aβ₄₂ is more prone to formation of insoluble aggregates. Jarrett et al. 1993 Cell 73:1055-1058, Further, mutations in APP which increase the relative proportion of Aβ₄₂ have been linked to familial AD. Suzuki et al. 1994 Science 264:1336-1340. In addition, Aβ₄₂ has been shown to be preferentially deposited in amyloid plaques. Iwatsubo et al. 1994 Neuron 13:45-53.

In vitro studies indicate the existence of at least two β-secretase pathways. In the endosomal/lysosomal pathway, APP targeted to the cell surface is endocytosed and delivered to endosomes and lysosomes where β- and γ-cleavages can occur (Golde et al. 1992 Science 255:728-730; Nordstedt et al. 1993 J. Biol. Chem. 268:608-612; Haass et al. 1992a Nature 357:500-503; Koo, E. H. and Squazzo, S. 1994 J. Biol. Chem. 269:17386- 17389; Lai et al. 1995 J. Biol. Chem. 270:3565-3573; Perez et al. 1996 J. Biol. Chem. 271:9100-9107). The alternative β- secretory pathway is believed to generate Aβ in Golgi-derived vesicles, most likely secretory vesicles, prior to secretion (Haass et al. 1995a Nature Med. 1:1291-1296; Higaki et al. 1995 Neuron 14:651-659; Perez et al. 1996 J. Biol. Chem. 271:9100- 9107; Thinakaran et al. 1996b J. Biol. Chem. 271:9390-9397).

Both Aβ₄₀G and Aβ₄₂ have been shown to be produced intracellularly from endogenous wild-type APP695 by cultured postmitotic CNS neuronal cells (NT2N) that are induced to differentiate from a human teratocarcinoma cell line (NT2) by treatment with retinoic acid (Pleasure et al. 1992 J. Neurosci. 12:1802-1815; Pleasure, S. J. and Lee, V. M. -Y. 1993 J. Neurosci. Res. 35: 585-602; Wertkin et al. 1993 Proc. Nat7. Acad. Sci. USA 90:9513-9517; Turner et al. 1996 J. 1iol. Chem. 271:8966-8970). To date, the human derived NT2N neuron is the only cell line documented to generate intracellular Aβ₄₀ and Aβ₂ before their eventual release into the medium (Turner et al. 1996 J. Biol. Chem. 271:8966-8970). Because neurons are the cell type most adversely affected by AD, the NT2N neurons represent a unique system for the study of APP processing and Aβ biogenesis. An essential first step in the analysis of such pathways is the identification of the proteolytic fragments that are the products of these cleavages.

It has now been found that γ-secretase acts in the Endoplasmic Reticulum (ER) to yield A+62₄₂. The N-terminal fragment generated by β-cleavage (i.e., APPβ) has also been found to be produced by β-secretase intracellularly in NT2N neurons prior to secretion. These protease activities were identified to occur in the Endoplasmic Reticulum (ER) /Intermediate Compartment (IC) of neuronal cells utilizing inhibition with Brefeldin A (BFA), incubation at 15° C., and expression of exogenous APP bearing the di-lysine ER-retrieval motif. Accordingly, the present invention relates to methods of identifying compounds targeted to the endoplasmic reticulum which increase or decrease processing of amyloid precursor protein into amyloid β peptides found in neuritic plaques and vascular deposits that accumulate in brains of patients with Alzheimer's disease.

### SUMMARY OF THE INVENTION

Novel β-secretase pathways have now been identified that produce amyloid βpeptides, Aβ₄₂ and APPβ, in the endoplasmic reticulum/intermediate compartment. Discovery of these enzymatic pathways and their location within the endoplasmic reticulum can be used to design new therapeutic approaches or agents which reduce production of amyloid β peptides. An object of the present invention is to provide a method of identifying agents which modulate processing of amyloid precursor protein into amyloid p peptides found in neuritic plaques and vascular deposits that accumulate in brains of patients with Alzheimer's disease. In one embodiment, the method of the present invention may be used to identify inhibitors of this process which may be useful in the treatment of Alzheimer's disease. Alternatively, the method of the present invention may be useful in identifying agents or genetic mutations which increase this process, thereby increasing the formation of amyloid β peptides and the possibility of developing Alzheimer's disease.

### DETAILED DESCRIPTION OF THE INVENTION

APP serves as a substrate for a variety of proteolytic processing pathways, only some of which result in the production of Aβ (Selkoe, D. J. 1994 Ann. Rev. Cell. Biol. 10:373-403) . However, AG is the major component of senile plaques in the AD brain. Moreover, mutations in the APP gene associated with Familial Alzheimer's Disease (FAD) alter APP processing and Aβ production in vitro (Citron et al. 1992 Nature 360:672-674; Cai et al. 1993 Science 259:514-516; Suzuki et al. 1994 Science 264:1336-1340). Thus, determination of the proteolytic events that lead to Aβ production and identification of the proteases responsible for each step as well as the sites of their action are important in the development of treatments for Alzheimer's disease and in the identification of causes for this disease.

In the present invention, the NT2N system was used to study APP processing in neurons. NT2N cells have been reported produce intracellular Aβ (Wertkin et al. 1993 Proc. Natl. Acad. Sci. USA 90:9513-9517; Turner et al. 1996 J. Biol. Chem. 271:896678970). NT2N neurons express the isoform of APP expressed almost exclusively in the CNS (i.e., APP695) and generate detectable intracellular levels of both Aβ₄₀ and Aβ₄₂. Further, they constitutively produce and secrete Aβ. Using antibodies specific to APPβ and other proteolytic fragments, a number of intracellular β-secretase activities which cleave on the amino terminus side of Aβ have now been identified and characterized. Moreover, novel β-secretase activities that occur in a pre-Golgi compartment have now been identified.

For example, β-secretase(s) cleaves APP at the amino terminus of the Aβ domain releasing a distinct N-terminal fragment (APPβ). To determine if intracellular APPβ could be recovered from NT2N cells, samples of cell lysate were immunoprecipitated with Karen (an antiserum raised to the N-terminal region of APP). The presence of APPβ in the immunoprecipitate was determined by immunoblot analysis using 53 (a polyclonal antibody specific for the free carboxy-terminus of APPβ). This polyclonal antibody detected a single band of approximately 95 kDa. The identity of this 95 kDa APP fragment to be APPβ cleaved at the β-secretase site was confirmed by: 1) the inability of 369W, an antibody specific for the C-terminus of APP, to recognize this fragment; 2) the inability of 6E10, an antibody specific for the first 10 amino acid residues of Aβ, to detect this fragment; 3) the binding of Karen, an antibody that recognizes all APP species, to this fragment; 4) the fact that this intracellular APP fragment is about 11-12 kDa smaller than APP_{FL}; and 5) the detection of the same 95 kDa APP fragment using a different antibody specific for APPβ (i.e., 192; Seubert et al. 1992 Nature 359:325-327). APPβ was readily detected in the media of NT2N neurons and co-migrated with APPβ recovered from the cell lysates thus indicating that APPβ was secreted.

The detection of intracellular APPβ and Aβ in NT2N neurons indicates that 0-secretase pathway including β- and γ-secretase occur in an intracellular compartment. The absence of intracellular APPα, however, suggests that the majority or all of the α-secretase activity occurs at a different site. To further confirm that the β-secretase pathways, but not the α-secretase pathway, occur inside these cells, the cell lysate of NT2N neurons was examined for the products of these respective pathways: Aβ, which is generated by β- and γ-secretase cleavages; and p3, a product of γ- and γ-secretase cleavages. In these experiments, cell lysates of metabolically labeled NT2N neurons were immunoprecipitated with monoclonal antibodies (mAb) that can distinguish between these peptides: 4G8 recognizes both Aβ and p3; Ban50, however, binds only to Aβ and not p3. Data from these experiments clearly demonstrates that Aβ, but not p3, is produced intracellularly. The p3 fragment was not detected in cell lysates even after prolonged exposure of the film. By contrast, both Aβ and p3 were readily recovered from the media.

A series of experiments were performed which confirm that APPβ is derived from APP_{FL} within the cell prior to secretion. In a first set of experiments, APPβ was recovered from NT2N cell lysates even after intact NT2N neurons were treated with trypsin. Cultures of NT2N neurons were treated with trypsin at 4° C. Under such conditions, cell surface-associated but not intracellular APPβ should be proteolyzed. However, a similar amount of APP: was recovered from NT2N neurons regardless of trypsin treatment. By contrast, when the NT2N neurons were treated with trypsin and 0.1 Triton X-100, intracellular APPβ was completely eliminated. Thus APPβ recovered from the NT2N cell lysate is indeed produced in an intracellular compartment.

A second set of experiments confirmed the continuous presence of steady state levels of APPβ in NT2N neurons, together with a delay in the detection of APPβ in freshly replenished medium. In these experiments, NT2N neurons were washed with fresh medium and the amount of intracellular as well as secreted APPβ and APPα were measured over an 8 hour period by immunoprecipitation of cell lysates and media with Karen followed by immunoblotting with either antibody 53 (for APPβ) or 6E10 (for APPα) . Secreted APPβ was first detected in 3 to 5 hours, and its accumulation in the medium continued over the 8 hour incubation period. In contrast, APPα was detected in one hour, suggesting that APPα is produced at a faster rate than APPβ. As seen with APPβ, APPβ accumulated in the conditioned media over time. A steady state level of APPβ was recovered from NT2N cell lysates prepared from parallel cultures over a period of 8 hours this indicating that intracellular APPβ is produced constitutively.

Pulse-chase experiments were also performed which demonstrated that the turnover of intracellular APPβ lags behind the turnover of newly synthesized APP_{FL,} thereby confirming that APPβ is generated from APP_{FL} inside NT2N neurons before secretion. This pulse-chase paradigm permits a more rigorous study of the temporal relationship between intracellular and secreted APPβ. In these experiments, NT2N cultures were pulsed with [³⁵S]methionine for one hour and then chased for different lengths of time. After one hour of chase time, full length APP (APP_{FL}) immunoprecipitated from the cell lysate began to decline while the intracellular level of APPβ continued to increase until 4 hours, after which it also declined. This lag in maximum production of intracellular radiolabeled APPβ indicates that APPβ is produced intracellularly from APP_{FL} by 9-secretase cleavage. Further, the one hour delay in the secretion of APPβ into the medium as well as the accumulation of this fragment with increasing chase time supports a temporal relationship between APPβ that is produced intracellularly and APPβ that is secreted into the medium.

The detection of APPβ in the cell lysate of NT2N neurons, together with the presence of Aβ₄₀ and Aβ₄₂ (Turner et al. 1996 J. Biol. Chem. 271:8966-8970), establish that an intracellular β-secretase pathway(s) exists in these cells. At present, no other cell line has been reported to produce detectable levels of intracellular APPβ from endogenous or over-expressed wild-type APP (Seubert et al. 1993 Nature 361:260-263; Haass et al. 1995a Nature Med. 1:1291-1296; Thinakaran et al. 1996b J. Biol. Chem. 271:9390-9397). Only human kidney 293 cells stably transfected with APPsw cDNA yield the related APPβsw fragment from the cell lysates (Haass et al. 1995a Nature Med. 1:1291-1296; Martin et al. 1995 J. Biol. Chem. 270:26727-26730). In these non-neuronal cells, however, treatment with BFA completely eliminates APPβsw and Aβ production (Haass et al. 1995a Nature Med. 1:1291-1296; Martin et al. 1995 J. Biol. Chem. 270:26727-26730; Essalmani et al. 1996 Biochem. Biophy. Res. Commun. 218:89-96). In contrast, NT2N neurons continue to produce APPβ and Aβ during treatment with BFA, implying that the subcellular site(s) of the β-secretase pathway is cell-type specific. In these experiments, NT2N neurons were metabolically labeled with [³⁵S]methionine in the presence or absence of 20 μg/ml Brefeldin A (BFA). BFA is a pharmacological agent that causes a redistribution of the Golgi into the ER. In the absence of BFA, APP_{FL}, APPβ, and Aβ were recovered from the cell lysates while APPα, APPβ, and Aβ were detected in the media of NT2N neurons. Surprisingly, in the presence of BFA, not only APP_{FL} but also APPβ, and Aβ continued to be recovered from NT2N cell lysates. The effectiveness of BFA was verified by the fact that the secretion of APPα, APPβ, and Aβ into the medium was completely abolished in its presence.

Cell lysates and medium from the cells were then analyzed using an ELISA system which quantitatively distinguishes between Aβ₄₀ and Aβ₄₂ to ascertain whether alteration of Aβ levels by BFA was the result of altered levels of Aβ₄₀, Aβ₄₂ or both. Suzuki et al. 1994 Science 264:1336-1340. Results from the ELISA correlated with the immunoprecipitation data in that BFA abolished secretion of Aβ into the medium and reduced overall expression of intracellular Aβ by approximately 60%. However, it was the complete loss of A+β₄₀ that accounted for this decrease. Aβ₄₂ was largely unaffected by BFA treatment. Thus, γ-secretases appear to be active in the endoplasmic reticulum, but only to yield Aβ₄₂. Further, it is believed that Aβ₄₂ is preferentially generated in the ER while Aβ₄₀, is generated in more distal components of the exocytic pathway.

Since BFA treatment results in retention of all newly synthesized proteins in the ER, additional experiments were conducted to confirm that the generation of Aβ₄₂ in the ER was not due to retention of newly synthesized proteases not being delivered to their proper site of action. Accordingly, an ER-retention signal was placed in APP695 in which the third and fourth amino acids from the C-terminus were changed to lysine (APP695_{ΔKK}) . This lysine motif is sufficient to retain heterologous transmembrane proteins in the ER and intermediate compartment. In these experiments, recombinant Semliki Forest virus (SFV) vectors to express APP695_{ΔKK. Previous studies have shown that despite high levels of SFV-mediated APP expression, SFV infected NT}2N cells display a high degree of fidelity in processing APP (Wertkin et al. 1993 Proc. Natl. Acad. Sci. USA 90:9513-9517; Turner et al. 1996 J. Biol. Chem. 271:8966-8970). Furthermore, it was found that cytopathic effects of SFV infection in NT2N cells as measured by LDH release do not develop until more than 48 hours after infection. To determine if APP695_{ΔKK} was in fact retained in the ER, indirect immunofluorescence microscopy was used to monitor both intracellular and cell surface distribution of SFV-expressed APP695 and APP695_{ΔKK}. While intracellular levels of APP695 and APP695_{ΔKK} appeared similar, only APP695 was expressed on the cell surface. BFA treatment blocked surface expression of APP695. In addition, APP695_{ΔKK} did not acquire resistance to endoglycosidase H digestion, a further indication that APP695_{ΔKK} is retained in the ER.

By retaining APP695_{ΔKK} in the ER, it was then determined whether Aβ is generated in this compartment without concomitant BFA-induced accumulation of other cellular proteins. SFV-infected NT2N cells were metabolically labeled overnight, and Aβ immunoprecipitated from the medium and cell lysate. It was found that ER retention of APP by the KK retention signal efficiently blocked Aβ secretion, yet failed to block all intracellular Aβ biosynthesis. Western blot analysis of intracellular and secreted APP showed that APP695 and APP695_{ΔKK were expressed to comparable levels. As with BFA treatment, cells expressing APP}695_{ΔKK} produced 40% of the total intracellular Aβ generated from APP695. Quantitative ELISA showed that this reduction was accounted for by the loss of Aβ,₄₀. Aβ₄₂ levels were not affected. Thus, Aβ₄₂ is cleaved from APP while still in the ER.

APPβ recovered from BFA treated cells was found to migrate with an accelerated electrophoretic mobility compared to APPβ from non-treated cells, suggesting that this fragment may have been derived from immature APP. To confirm that incomplete maturation of APP was the cause of the shift in electrophoretic mobility of the APP, fragment generated in the presence of BFA, NT2N cells were metabolically labeled with [³⁵S]methionine in the presence or absence of BFA. APPβ immunoprecipitated from the cell lysate was then incubated with N-glycosidase F (Nglyc F), an enzyme that removes N-linked carbohydrate chains. APPβ from BFA-treated NT2N neurons migrated more quickly than APPβ recovered from untreated cells. After digestion with Nglyc F, APPβ demonstrated a mobility downshift in SDS-PAGE. However, APPβ from BFA treated cells still migrated faster than APPβ from non-treated cells despite enzymatic removal of all N-linked carbohydrate chains. Thus, the increased electrophoretic mobility of APPβ in the presence of BFA cannot be accounted for solely by differences in N-linked carbohydrate processing.

In addition to N-linked glycosylation, however, APP undergoes a variety of post-translational modifications, including the addition of O-linked carbohydrate chains. Therefore, both N- and O-linked carbohydrate chains were removed from immuno-precipitated APPβ by simultaneous digestion with Nglyc F, O-glycosidase, and neuraminidase. Fully deglycosylated APPβ co-migrated with APPβ recovered from BFA treated NT2N neurons. Furthermore, combined BFA inhibition and deglycosylation did not induce a greater mobility shift than either of these treatments alone. Accordingly, APPβ generated from BFA treated NT2N neurons is believed to represent β-secretase processing of immature (non-glycosylated) APP_{FL} in a pre-Golgi compartment.

To further verify that β-secretase cleavage occurs early in the biosynthetic pathway of NT2N neurons, an alternative non-pharmacological method to block protein transport from the ER to the Golgi was employed. Incubation of cultured cells at 15° C. has been shown to inhibit newly synthesized proteins from exiting the intermediate compartment (Saraste, J. and Kuismanen, E. 1984 Cell 38:535-549; Saraste et al. 1986 Proc. Natl. Acad. Sci. USA 83:6425-6429; Schweizer et al. 1990 Eur. J. Cell. Biol- 53:185-196). In these experiments, NT2N cells were incubated at 15° C. for 16 hours. Only the immature form of APP_{FL} was present after a 16 hour incubation at 15° C. as indicated by its sensitivity to Endoglycosidase H (Endo H) digestion, suggesting that it is not transported to the Golgi apparatus under these conditions. By contrast, incubation of the NT2N cells at 37° C yielded both immature and fully processed APP_{FL}. As expected, the immature APP_{FL} was Endo H sensitive, while the mature forms of APP_{FL} having acquired post-translational modifications after exiting the ER, were Endo H resistant. In addition, secreted forms of APP were not detected in cells maintained at 15° C., further substantiating the effectiveness of the temperature block. Significantly, continuous production of intracellular APPβ was observed at 15° C. despite the fact that the secretion of APP ectodomain is completely abolished. Thus, these data also support the endoplasmic reticulum (ER) /intermediate compartment (IC) of NT2N neurons as a β-cleavage site.

The processing of wild-type APP695 and APP695 bearing the ER retrieval motif (APP695_{ΔKK}; Jackson et al. 1990 EMBO J. 9:3153-3162; Jackson et al. 1993 J. Cell Biol. 121:317-333) in the NT2N cells was also examined with regard to cleavage of APPβ. To determine whether or not APPβ can be produced from APP695 _{ΔKK} wild-type APP695 and APP695_{ΔKK} were separately expressed in NT2N neurons by infection with SFV vectors bearing these constructs. Following infection, duplicate wells containing wild-type APP695 infected cells were also treated with 20 ,μ/ml BFA. The [³⁵S]methionine labeled cell lysates and the media were then sequentially immunoprecipitated with the antibodies 53 and Karen. Only the immature form of APP_{FL} was detected from cells expressing APP695_{ΔKK}. Significantly, intracellular production and secretion of APPβ was not affected by genetic targeting of APP to the ER. Furthermore, it was found that unlike inhibition with BFA that eliminates transport of all proteins from the ER to the Golgi, specific retrieval of full-length APP695+_{ΔKK} to the ER allowed the APPβ fragment generated in the ER/IC to be transported to the Golgi complex for modification before secretion. Thus, it is believed that once the ER retention motif is cleaved from the APPβ fragment, it can then be transported to the Golgi complex for further maturation and subsequent secretion.

Generation of Aβ in the ER of NT2N neurons identifies these cells as a unique system in which to study amyloidogenic processing of APP and its role in the pathogenesis of AD. Mutations in both the APP gene and the recently identified presenilin genes are believed to cause AD by altering APP processing in ways that lead to the production of more amyloidogenic forms of Aβ (i.e., Aβ₄₂; Scheuner et al. 1996 Nature Med. 2:864-870). Recently, in both non-neuronal and neuronal cells (including the NT2N neurons used in this study), the presenilin proteins have been localized to the ER (Cook et al. 1996 Proc. Natl. Acad. Sci. USA 93:9223-9228; Kovacs et al. 1996 Nature Med. 2:224-229; Thinakaran et al. 1996a. Neuron 17:181-190). Thus, the identification of amyloidogenic processing that occurs within the ER of neurons suggests that direct or indirect interaction may occur between the presenilins and APP. Furthermore, the mutations in the presenilin genes may alter this interaction in a manner that leads to increased production of APPβ and ultimately Aβ₂. Accordingly, the identification of these secretase pathways in the ER of neuronal cells will permit the examination of the effects of both FAD-linked mutations occurring in the APP as well as the presenilin genes on the processing of APP in the ER. Further agents which modulate APPβ processing by increasing or decreasing production of APPβ and Aβ₄₂ can be identified by determining their effects on levels of APPβ and Aβ₄₂ produced by β- and γ-secretases in the ER of neuronal cells such as NT2N cells. Levels of peptides produced by this pathway can be routinely determined in accordance with teachings provided herein. Agents identified by this method to inhibit levels of APPβ and/or Aβ₄₂ produced by these enzymatic pathways may be useful in treating Alzheimer's disease while agents which increase levels of APPβ produced by this pathway may be causative factors in the development of Alzheimer's disease.

The following nonlimiting examples are provided to further illustrate the present invention.

### EXAMPLES

### Example 1 Cell Culture

NT2 cells derived from a human embryonal carcinoma cell line (Ntera 2/c1.D1) were grown and passage twice weekly in Opti-Mem (Life Technologies, Inc.) supplemented with 5% fetal bovine serum (FBS) and penicillin/streptomycin (P/S) in accordance with procedures described by Pleasure et al. 1992 J. Neurosci. 12:1802-1815 and Pleasure, S. J. and Lee, V. M. -Y. 1993 J. Neurosci. Res. 35:585-602. To begin differentiation, 2.5×106 cells were seeded in a 75 cm² (T75) flask and fed with Dulbecco's modified Eagle's medium (DMEM) HG (Life Technologies, Inc., Gaithersburg, MD) containing 10 μM retinoic acid, 10% FBS and P/S twice weekly for 5 weeks. The cells in a single T75 flask were then replated at a lower density in 2×225 cm² (T225) flasks for 10 days (Replate 1 cells). NT2N neurons with greater than 99% purity were then obtained by enzymatic treatment and mechanical dislodgment of Replate 1 cells and replated at a density of 6×106 cells per 10 cm dish previously coated with polylysine and Matrigel as described by Pleasure et al. 1992 J. Neurosci. 12:1802-1815. The NT2N neurons were maintained in medium consisting of one part conditioned medium and one part DMEM HG containing 10% FES and P/S. For experiments involving the incubation of NT2N neurons at 15° C. for 16 hours, regular medium containing DMEM HG and 10% FES was replaced by DMEM HG containing 25 mM HEPES, 10% FBS, and P/S. Cultures of NT2N neurons were used for experiments when they were between 3 to 4 weeks old. CHO695 cells were grown and passaged three times per week in α-MEM (Life Technologies, Inc., Gaithersburg, Md.) supplemented with 10% FBS and P/S. M17 cells were grown and passage once per week in Opti-mem (Life Technologies, Inc., Gaithersburg, Md.) containing 10% iron enriched calf serum and P/S.

### Example 2 Metabolic Labeling, Gel Electrophoresis, Immunoblotting and Quantitation

Cultured NT2N neurons were starved in methionine-free DMEM HG (Life Technologies, Inc., Gaithersburg, Md.) for 30 minutes prior to incubation in fresh methionine-free DMEM HG containing 0.5 mCi/ml of [³⁵S]methionine (sp act. 1000 Ci/mmol; NEN-DuPont, Boston, Mass.). For steady-state labeling studies, NT2N neurons were labeled with [³⁵S]methionine continuously for 16 hours. For pulse-chase studies, cells were labeled with [³⁵]methionine for 1 hour, washed twice with methionine-containing DMEM, and then chased in the same medium for 0 to 24 hours. APP_{FL}, APPα and APPβ were separated on 7.5% Laemmli SDS-PAGE gels, and Aβ and p3 were separated on 10/16.5% step-gradient Tris-Tricine gels. These gels were either stained with Coomassie Brilliant Blue R (Pierce, Rockford, Ill.) and dried or transferred to nitrocellulose membranes and dried prior to exposure on PhosphorImager plates (Molecular Dynamics Inc., Sunnyvale, Calif.) for 3-5 days. The nitrocellulose replicas containing the immunoprecipitates were further probed with different antibodies in accordance with procedures described by Wertkin et al. 1993 Proc. Natl. Acad. Sci. USA 90:9513-9517. Quantitation of bands in the autoradiogram was performed using the ImageQuant software (Molecular Dynamics Inc. Sunnyvale, Calif.) in accordance with procedures described by Turner et al. 1996 J. Biol. Chem. 271:8966-8970. Radiolabeled proteins in SDS-PAGE gels and nitrocellulose replicas were also analyzed by standard autoradiographic methods. All experiments were repeated between 3 and 6 times.

### Example 3 Sample Preparation and Serial Immunoprecipitations

Cell lysates were prepared in accordance with procedures described by Golde et al. 1992 Science 255:728-730. Protein concentration was determined by the bicinchoninic acid procedure (Pierce, Rockford, Ill.). Media were centrifuged at 100,000×g for one hour at 4° C. before immunoprecipitation. Both cell lysates and media were precleared with protein A-Sepharose (Pharmacia Biotech, Piscataway, N.J.) in RIPA for one hour at 4° C. After recentrifugation at 15,000×g for one minute, the supernatants were rocked overnight at 4° C. with fresh protein A-Sepharose and the appropriate primary antibody. After collecting the immunoprecipitates by recentrifugation at 15,000×g for 1 minute, the supernatants were used in a second round of immunoprecipitation with fresh protein A-Sepharose and a different primary antibody.

### Example 4 Trypsin Treatment of NT2N Neurons

NT2N neurons were metabolically labeled with 0.5 mCi/ml [³⁵S] methionine for 16 hours in accordance with the procedure set forth in Example 2. After rinsing the cultures twice with PBS, the NT2N neurons were incubated on ice for 20 minutes with PBS, with 10 μ/ml of trypsin in PBS alone (Life Technologies, Inc., Gaithersburg, Md.), or with 10 μg/ml trypsin and 0.1% Triton X-100 in PBS. Following this treatment, trypsin was inactivated by the addition of 100 μg/ml soybean trypsin inhibitor. The cells were then washed with PBS, scraped into cell lysis buffer, and processed for immunoprecipitation as described in Example 3.

### Example 5 Brefeldin A Treatment of NT2N Neurons and Deglycosylation of Immunoprecipitated APPμ

NT2N neurons were pretreated with 20 Ag/ml of BFA for 1 hour before the addition of 0.5 mCi/ml of [³⁵S] methionine to the cultures for 16 hours in the absence or presence of BFA. The cell lysates and media were processed for immunoprecipitation as described in Example 3. For deglycosylation of APPG, the immunoprecipitates containing APPβ were washed twice in sodium phosphate buffer (20 mmol/l, pH 7.2) and boiled for 2 minutes in 10 μl of 1% SDS. The samples were then boiled for an additional 2 minutes after adding 90 μl of the sodium phosphate buffer with sodium azide (10 mmol/l), EDTA (50 mmol/l), and n-Octylglucoside (0.5% w/v). After this denaturation step, deglycosylation was initiated by the addition of 2 mU Neuraminidase (Arthrobacter; Boehringer Mannheim, Indianapolis, Ind.), 2.5 mU 0-Glycosidase (Boehringer Mannheim, Indianapolis, Ind.), and 0.4 U N-Glycosidase F (Boehringer Mannheim, Indianapolis, IN). The samples were then incubated at 37° C. for 18 hours and deglycosylated APPβ was run on 7.5% SDS-PAGE gels as described in Example 2.

For Endoglycosidase H (Endo H) sensitivity test, cell lysates and media were immunoprecipitated with Karen in accordance with procedures described in Example 3. Immunoprecipitates were recovered in 100 Ml 60 mM phosphate buffer, pH 5.7 with 1t SDS. The samples were then split in half (50 μl each), and incubated with 4 μl Endo H (Boehringer Mannheim, Indianapolis, IN) or vehicle at 37° C. for 18 hours. The samples were then run on 7.5w SDS-PAGE gels in accordance with procedures described in Example 2.

### Example 6 Antibodies for Immunoprecipitation and Immunoblotting

Karen is a goat polyclonal antisera raised to the large secreted N-terminal fragment of APP, and antibody 53 is a rabbit polyclonal antisera raised to a synthetic peptide corresponding to the amino acid sequence SEVKM. Antibody 53 binds specifically to the free C-terminus of APPβ as disclosed by Howland et al. 1995 Neurobiol. Aging 16:685-699. Antibody 369W is a rabbit polyclonal antiserum raised to a synthetic peptide corresponding to the last 45 amino acid residues at the C-terminus of APP. Also used in this study were three monoclonal antibodies (MAb) to Aβ that are specific for residues 1-17 (6E10; Kim et al. 1988 Neurosci. Res. Conmuun. 2:121-130); residues 1-10 (Ban5O; Suzuki et al. 1994 Science 264:1336-1340) and residues 18-25 (4G8; Kim et al. 1988 Neurosci. Res. Commun. 2:121-130).

### Example 7 Preparation of SFV bearing pSFV-1(APP695) and pSFV-1 (APP695,K)

The di-lysine motif was introduced into APP695 by standard PCR site-directed mutagenesis of pSFV-1(APP695) using primers 5′-CGAAAACCACCGTGGAGCTCCTT-3′(SEQ ID NO: 1) and 5′- TTAACCCGGGCTAGTTCTGCTTCTTCTCAAAGAACTTGT-3′ (SEQ ID NO: 2). The mutation containing PCR fragment was isolated by digestion with Bsm-1 and Xma-1, then ligated into pSFV(APP695) to yield pSFV(APP695_{ΔKK}) . All pSFV-1 constructs, including a pSFV helper plasmid with SFV structural genes, were linearized by digestion with Spe-1 and then used as a template for RNA synthesis with SP6 RNA polymerase. Co-electroporation of RNA from the expression and helper plasmids into BHK cells yielded infectious, replication-defective virus that was harvested 24 hours later in accordance with procedures described by Liljestrom, P. and Garoff, H. 1991 Bio/Technology 9:1356-1361. Accurate determination of viral stock titers were made in accordance with procedures described by Cook et al. 1996 Proc. Natl. Acad. Sci. USA 93:9223-9228. For all infection experiments approximately 1×10⁶ NT2N neurons per 35 mm dish were infected in serum free medium at a multiplicity of infection (MOI) of 7-10. When called for, 20 μg/ml BFA was added after the completion of the infection step.

What is claimed is:

1. A method of identifying agents which increase or decrease processing of amyloid precursor protein into amyloid β peptides found in neuritic plaques and vascular deposits that accumulate in brains of patients with Alzheimer's disease comprising:
contacting NT2N cells with a compound or agent suspected of increasing or decreasing amyloid precursor protein processing; and
measuring levels of amyloid P peptides formed in the endoplasmic reticulum of the cells.

2. A method of diagnosing Alzheimer's disease in a patient comprising detecting in the patient an agent identified to increase processing of amyloid precursor protein into amyloid β peptides found in neuritic plaques and vascular deposits that accumulate in brains of patients with Alzheimer's disease in accordance with the method of claim 1.

3. A method of inhibiting processing of amyloid precursor protein into amyloid β peptides found in neuritic plaques and vascular deposits that accumulate in brains of patients with Alzheimer's disease comprising administering to the patient an agent which decreases processing of amyloid precursor protein into amyloid β peptides found in neuritic plaques and vascular deposits that accumulate in brains of patients with Alzheimer's disease wherein said agent is identified in accordance with the method of claim 1.


SEQUENCE LISTING (2 sequences)

SEQ ID NO: 1 - DNA, 23 bp, Artificial Sequence
  Description of Artificial Sequence  Synthetic
cgaaaaccaccgtggagctcctt

SEQ ID NO: 2 - DNA, 39 bp, Artificial Sequence
  Description of Artificial Sequence  Synthetic
ttaacccgggctagttctgcttcttctcaaagaacttgt